(19) United States
(12) Patent Application Publication
(10) Pub. No.: US 2022/0088406 A1
(43) Pub. Date: Mar. 24, 2022

(54) Eyebrow Treatment Device

(71) Applicant: Johann Verheem, Carlsbad, CA (US)

(72) Inventor: Johann Verheem, Carlsbad, CA (US)

(21) Appl. No.: 17/024,703

(22) Filed: Sep. 18, 2020

(51) Int. Cl. A61N 5/06; A61H 23/00; A61N 1/36; A61F 7/02; A61M 35/00

(52) U.S. Cl. CPC A61N 5/0617; A61H 23/00; A61N 1/36014; A61N 5/062; A61F 2007/0003; A61M 35/10; A61M 2205/051; A61N 2005/0647; A61F 7/02

(57) ABSTRACT

A treatment device worn on top and around a user's eyebrows providing therapeutic benefits.

### CROSS-REFERENCE TO RELATED APPLICATIONS

This application is a United States non-provisional application which is related to U.S. provisional patent application No. 62/875,921 entitled “Treatment Device for Eyebrows” and filed Jul. 18, 2019, as well as U.S. provisional patent application No. 62/926,478 entitled “Eye Brow Device” and filed on Oct. 26, 2019, the contents of which is incorporated herein by reference in its entirety as if set forth verbatim.

### FIELD

The following description relates generally to a treatment device and method that is used to treat human eyebrows. The treatment device and method consist of a physical treatment device that is placed on the user's eyebrows to provide cosmetic, therapeutic and medical benefits. The treatment device can be used in conjunction with or without soluble ingredients that will be absorbed by the hair and follicles of the eyebrows or skin around the eyebrows. Such soluble ingredients can be delivered to the eyebrows and skin surrounding the eyebrows where the soluble ingredient or ingredients is applied directly to the eyebrows and skin around the eyebrows or by means of a patch, mask or other physical material that is impregnated with the soluble ingredient or ingredients and placed on top of the eyebrows and skin around the eyebrows where the ingredient or ingredients is to be delivered. In addition, the treatment device can contain electronic components that provide therapeutic heat, light for phototherapy or vibration to treat the eyebrows. Furthermore, the treatment device can be made from a material that can be heated or cooled by for instance a microwave or a fridge prior to use in order to use heat and cold as enhancements of the treatment or therapy.

### BACKGROUND

Applying cosmetic and therapeutic ingredients to the eyebrows is an age-old custom. Cosmetics enhance and accentuate the appearance of the eyebrows considered desirable for centuries. At the same time, therapeutic topical ingredients such as ointments, creams and lotions have been used to treat cracked eyebrows in an effort to enhance the appearance of the eyebrows

The current invention provides a novel treatment device that provides a physical treatment device that is worn by the user on their eyebrows. The treatment device can be made from rigid, semi-rigid or flexible material and can provide benefits by providing cold, heat, vibration and phototherapy to the eyebrows. In addition, the treatment device can be used in conjunction with soluble ingredients that will be absorbed by the user's eyebrows and skin around the eyebrows.

### BRIEF DESCRIPTION OF THE DRAWINGS

Embodiments of the present technology will be described and explained through the use of the accompanying drawings. The same reference numbers in different drawings may identify the same or similar elements.

FIG. 1 is a front perspective of a representative embodiment of the device

FIG. 2 is a front perspective of a representative embodiment of the device

The drawings, some components and/or operations can be separated into different blocks or combined into a single block when discussing some embodiments of the present technology. Moreover, while the technology is amenable to various modifications and alternative forms, specific embodiments have been shown by way of example in the drawings and are described in detail below. The intention, however, is not to limit the technology to the particular embodiments described herein. On the contrary, the technology is intended to cover all modifications, equivalents, and alternatives falling within the scope of the technology as defined by the appended claims.

### DETAILED DESCRIPTION

The features of the presently disclosed solution may be economically molded or assembled by using one or more distinct parts and associated components which, may be assembled together for removable or integral application. Unless defined otherwise, all terms of art, notations and other scientific terms or terminology used herein have the same meaning as is commonly understood by one of ordinary skill in the art to which this disclosure belongs.

In some cases, terms with commonly understood meanings may be defined herein for clarity and/or for ready reference, and the inclusion of such definitions herein should not necessarily be construed to represent a substantial difference over what is generally understood in the art. All patents, applications, published applications and other publications referred to herein are incorporated by reference in their entirety. If a definition set forth in this section is contrary to or otherwise inconsistent with a definition set forth in the patents, application, published applications and other publications that are herein incorporated by reference, the definition set forth in this section prevails over the definition that is incorporated herein by reference.

As used herein, “a” or “an” means “at least one” or “one or more.” As used herein, the term “user”, “subject”, “end-user” or the like is not limited to a specific entity or person. For example, the term “user” may refer to a person who uses the systems and methods described herein, and frequently may be a technician. However, this term is not limited to end users or technicians and thus encompasses a variety of persons or entities who can use the disclosed systems and methods.

As used herein, the term “treatment device” is used herein with reference to a device providing methods and components for medical and cosmetic treatment of eyebrows.

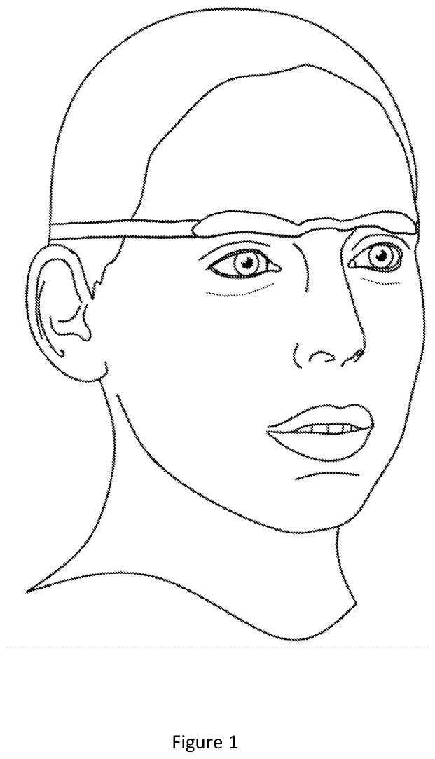

The disclosed solution can now be better understood by turning to the following detailed description. FIG. 1 shows the treatment device in one embodiment. In this embodiment the treatment device consists of a single rigid, semi-rigid or flexible shape that is placed across the user's eyebrows so that it can deliver treatment to the user's eyebrows. The treatment device is worn in this position for a period of time. In one embodiment the treatment device will adhere to the outside of the eyebrows through removable glue, similar to a Band-aid, or through suction. In another embodiment a strap, that wraps around the user's head and pulls the treatment device against the face, can also be used to hold the treatment device intact.

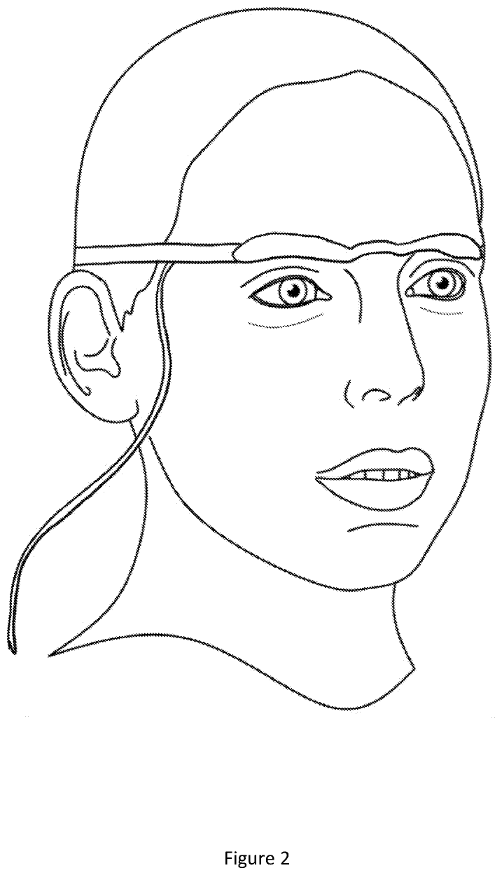

FIG. 2 show another embodiment of the device where there is an external power cord that can be plugged into a smartphone, tablet, USB port, electrical outlet, battery pack or other external sources of power.

One embodiment of the treatment device is made from materials such as silicone or gel that retains temperature well and can be heated or cooled through an external source such as a fridge or a microwave in order to provide therapy.

The treatment device can provide cosmetic and therapeutic benefits by itself when heated or cooled. The treatment device can also provide benefits when used in conjunction with a cream, lotion, gel or other soluble ingredient. In this embodiment a user will apply one or more soluble ingredient on their eyebrows or on the inside of the treatment device. This can include creams, lotions, gels and other cosmetic or therapeutic ingredients. The inside of the curved surface that wraps around the eyebrows and provides a space or cavity for inclusion of the soluble ingredients such as liquids, gels, creams or ointments against the eyebrows of the user. This method enables the treatment device to encapsulate the soluble ingredient close to the skin for effective absorption over a period of time without exposing the user's eyes to the ingredient. When heated or cooled, this method of wearing the treatment device while using a soluble ingredient can further enhance therapeutic and cosmetic benefits. In another embodiment the soluble ingredient or ingredients can be delivered to the eyebrows and skin around the eyebrows by means of a patch, mask or other physical material that is impregnated with the soluble ingredient or ingredients and placed on top of the eyebrows and skin around the eyebrows with the treatment device placed on top of such patch, mask or other physical material.

The treatment device can be worn for extended periods of time, even overnight. This compares to typical medical or cosmetic ingredients that are applied to the eyebrows and typically absorbs in its entirety in as little as 20 minutes. This form of inclusion enhances the total volume of liquid ingredient that the eyebrows will absorb and can result in larger, more hydrated eyebrows.

In another embodiment the treatment device contains one or more electronic lights providing phototherapy to the eyebrows and the skin around the eyebrows. In this embodiment, one or more light sources are placed, attached or molded inside or on top of the treatment device. In one embodiment several lights are molded or placed in the treatment device, projecting light on the eyebrows and skin around the eyebrows when worn. A light source inside the treatment device can use one or more wave lengths of light, such as infrared light or blue light or both, to provide the benefits typically associated with photo therapy. This embodiment of the treatment device also allows for a method of using the treatment device in conjunction with soluble ingredients for simultaneous or complementary benefits that further enhances the treatment of the eyebrows.

In another embodiment of the treatment device, the treatment device contains one or more electrical vibrating motors that enable movement of the skin's surface while the user is wearing the treatment device, providing a therapeutic massage while the topical ingredient is being absorbed by the skin. This embodiment can include or exclude phototherapy.

In one embodiment of the device the power source for the device is embedded in the band or other attachment to the device. In another embodiment of the device, power is provided through an external power cord that can be connected to a battery or smart phone to provide power to the unit.

What is claimed:

1. A treatment device worn on or around the eyebrows of a user that is worn on top of one or more soluble ingredients such as ointments, gels and creams.

2. A treatment device that is worn on or around the eyebrows of a user that contains a light source and electrical power source providing light therapy to the follicles of the eyebrows, the hair of the eyebrows and skin around the eyebrows.

3. A treatment device as described in claim 1, that also contains a light source and electrical power source providing phototherapy to the follicles of the eyebrows, the hair of the eyebrows and skin around the eyebrows.

4. A treatment device as described in claim 2 where the power source is external to the device and connected to the device with a power cord that can be connected to a smartphone, tablet, external battery pack or electrical outlet.

5. A treatment device as described in claim 3 where the power source is external to the device and connected to the device with a power cord that can be connected to a smartphone, tablet, external battery pack or electrical outlet.

6. A treatment device as described in claim 1 that provides vibration or electric muscle stimulation to increase blood flow and provide skin, muscle and tissue enhancing benefits to the user's eyebrows.

7. A treatment device as described in claim 2 that provides vibration or electric muscle stimulation to increase blood flow and provide skin, muscle and tissue enhancing benefits to the user's eyebrows.

8. A treatment device as described in claim 3 that provides vibration or electric muscle stimulation to increase blood flow and provide skin, muscle and tissue enhancing benefits to the user's eyebrows.

9. A treatment device as described in claim 1 that contains portions of material that can be heated or chilled in order to provide hot or cold therapeutic benefits to the wearer.

10. A treatment device as described in claim 2 that contains portions of material that can be heated or chilled in order to provide hot or cold therapeutic benefits to the wearer.

11. A treatment device as described in claim 3 that contains portions of material that can be heated or chilled in order to provide hot or cold therapeutic benefits to the wearer.

12. A flexible, soft or semi rigid treatment device made from a material that can be impregnated with a soluble ingredient that is worn on the eyebrows of a user where the soluble ingredient is dispensed from the treatment device to the eyebrows and the skin around the eyebrows.

13. A treatment device as described in claim 1 where the soluble ingredient is contained in a patch, mask or other physical material that is impregnated with such soluble ingredient or ingredients and placed on top of the eyebrows and skin around the eyebrows between the device and the eyebrows.

14. A treatment device as described in claim 2 where the soluble ingredient is contained in a patch, mask or other physical material that is impregnated with such soluble ingredient or ingredients and placed on top of the eyebrows and skin around the eyebrows between the device and the eyebrows.

15. A treatment device as described in claim 3 where the soluble ingredient is contained in a patch, mask or other physical material that is impregnated with such soluble ingredient or ingredients and placed on top of the eyebrows and skin around the eyebrows between the device and the eyebrows.
